(19) United States
(12) Patent Application Publication
(10) Pub. No.: US 2017/0027120 A1
(43) Pub. Date: Feb. 2, 2017

(54) LOW FLOW POND FOR ALGAE CULTIVATION

(71) Applicant: SAPPHIRE ENERGY, INC., SAN DIEGO, CA (US)

(72) Inventors: MEHRAN PARSHEH, LAS CRUCES, MN (US); MARCOS DELGADO, LAS CRUCES, MN (US); BRYN DAVIS, LAS CRUCES, MN (US); ALEXANDER THOMAS DIFFLEY, LAS CRUCES, MN (US)

(21) Appl. No.: 15/301,395

(22) PCT Filed: Mar. 19, 2015
(86) PCT No.: PCT/US2015/021599

§ 371 Date: Oct. 2, 2016

Related U.S. Application Data
(60) Provisional application No. 61/978,666, filed on Apr. 11, 2014.

(51) Int. Cl. A01G 33/00; A01H 4/00

(52) U.S. Cl. CPC A01G 33/00; A01H 4/001

(57) ABSTRACT

Disclosed herein is a system for circulating fluid in an algae cultivation pond. The system includes an algae cultivation pond having an aspect ratio of length to width between 0.7:1 to 2:1, and the pond is at least 5 acres in overall size. An island has a first side and a second side and is positioned in the pond to create a first channel adjacent the first side of the island and a second channel adjacent the second side of the island. An energy input means is located in the first channel and moves fluid around the island through the first channel and through the second channel. The second channel is at least three times wider than the first channel, and a length of the island is at least six times larger than a width of the island.

### BACKGROUND

Large ponds are used to cultivate algae where the cultivated algae may then be used in the production of biofuel. The ponds are commonly shaped as center pivot ponds, serpentine ponds, no-flow (or unmixed) rice field ponds or raceway ponds. In these ponds, the algae, water and nutrients circulate around the pond with typically paddlewheels providing the flow. Algae are kept suspended in the water, and are circulated back to the surface on a regular frequency. The ponds are normally shallow allowing sunlight to penetrate the water enabling the algae to be exposed to sunlight for the photosynthesis process. The ponds are operated in a continuous manner, with carbon dioxide and nutrients being constantly fed to the ponds, while algae-containing water is removed at the other end for processing.

Techno-economic analysis explores the relationships between the technical and economic aspects of a process or system. It provides a means to evaluate the technical and economic viability of a system, quantifying the trade-offs between system characteristics and identifying research priorities and targets. Traditional algae cultivation ponds do not meet techno-economic requirements for large scale algae production. Additionally, traditional cultivation ponds are expensive to construct and operate.

### SUMMARY

Disclosed herein is a system for circulating fluid in an algae cultivation pond. The system includes an algae cultivation pond having an aspect ratio of length to width between 0.7:1 to 2:1, and the pond is at least 5 acres in overall size. An island has a first side and a second side and is positioned in the pond to create a first channel adjacent the first side of the island and a second channel adjacent the second side of the island. An energy input means is located in the first channel and moves fluid around the island through the first channel and through the second channel. The second channel is at least three times wider than the first channel, and a length of the island is at least six times larger than a width of the island.

The present invention is better understood upon consideration of the detailed description below in conjunction with the accompanying drawings and claims.

### BRIEF DESCRIPTION OF THE DRAWINGS

FIG. 1 depicts an example embodiment for a low flow (LF) pond;

FIG. 2 is an example process flowchart for creating an LF pond;

FIG. 3 is a table with example simulated cases of various sized LF ponds;

FIG. 4 illustrates an example simulation for potential deadzones in LF ponds;

FIG. 5 shows an example embodiment of a harvest sub-pond located in an LF pond;

FIG. 6 is an example embodiment of a harvest sub-pond;

FIG. 7 illustrates an example embodiment of flow within a harvest sub-pond;

FIG. 8 is an example embodiment of an algae froth floatation harvest method in an LF pond; and

FIG. 9 is an example embodiment of an algae froth floatation harvest method in a harvest sub-pond.

### DETAILED DESCRIPTION

Disclosed herein is a system for circulating fluid in an algae cultivation pond. The system includes an algae cultivation pond having an aspect ratio of length to width between 0.7:1 to 2:1. The pond is at least 5 acres in overall size. An island has a first side and a second side and is positioned in the pond to create a first channel adjacent the first side of the island and a second channel adjacent the second side of the island. An energy input means is located in the first channel and moves fluid around the island through the first channel and through the second channel. The second channel is at least three times wider than the first channel, and a length of the island is at least six times larger than a width of the island.

In various embodiments, the energy input means may be in a single area in the first channel. The single area may be a point or along a single axis. The energy input means may be a pump, paddlewheel, fan, nozzle or jets. The fluid may have consistent flow through the second channel. The average fluid velocity may be at least 3 centimeters/second.

The average pond depth may be at least 15 inches. The pond may be at least 20 acres, 40 acres, 80 acres, 100 acres or 160 acres in overall size, and may have an aspect ratio of length to width between 0.8:1 to 1.2:1. The pond may be configured with clipped corners.

The island may be substantially elongated diamond shaped or oval shaped and may include substantially a pointed tip at both the first end and the second end. In one embodiment, the ratio of the length of the island to the width of the island may be approximately 3.75. In another embodiment, the ratio of the length of the island to the width of the island may be between 2 and 5.

In one embodiment, the pond may be configured to include harvesting corners for algae. The harvesting corners may be a semi-isolated region promoting advanced settling and concentration of algae. The density of an algae culture entering the harvesting corners may be greater than the density of the algae culture exiting the harvesting corners. The harvesting corners may be comprised of weirs. The weirs may create semi-isolated regions from a main body of the pond, and in one embodiment, may have a height of approximately 2 inches less than the average depth of the main body of the pond. The fluid with an algae culture may be exchanged between the main body of the pond and the harvesting corners over a top of the weirs.

Also disclosed herein is a method for circulating fluid in an algae cultivation pond. The method includes creating an algae cultivation pond having an aspect ratio of length to width between 0.7:1 to 2:1. The pond is at least 5 acres in overall size. An island with a first side and a second side is positioned in the pond forming a first channel adjacent the first side of the island, and a second channel adjacent the second side of the island. An energy input means is located in the first channel for moving fluid around the island through the first channel and through the second channel. The second channel is at least three times wider than the first channel, and a length of the island is at least six times larger than a width of the island.

Low flow (LF) ponds are a class of ponds, designed around the economic needs of large scale algae cultivation. This pond may be operated using different types of motivation systems such as paddlewheels, jet nozzle systems, direct centrifugal and submersible pumping or a combination of these systems. The basic concept is to minimize build costs of the configuration while accommodating an efficient means of circulating fluid, such as water, with low energy input. In an LF pond, there is a motive fluid, for example, a portion of the pond water, which is used to cause the circulation flow of the entire pond fluid without requiring the entire volume of the pond fluid to pass through the system at a constant rate.

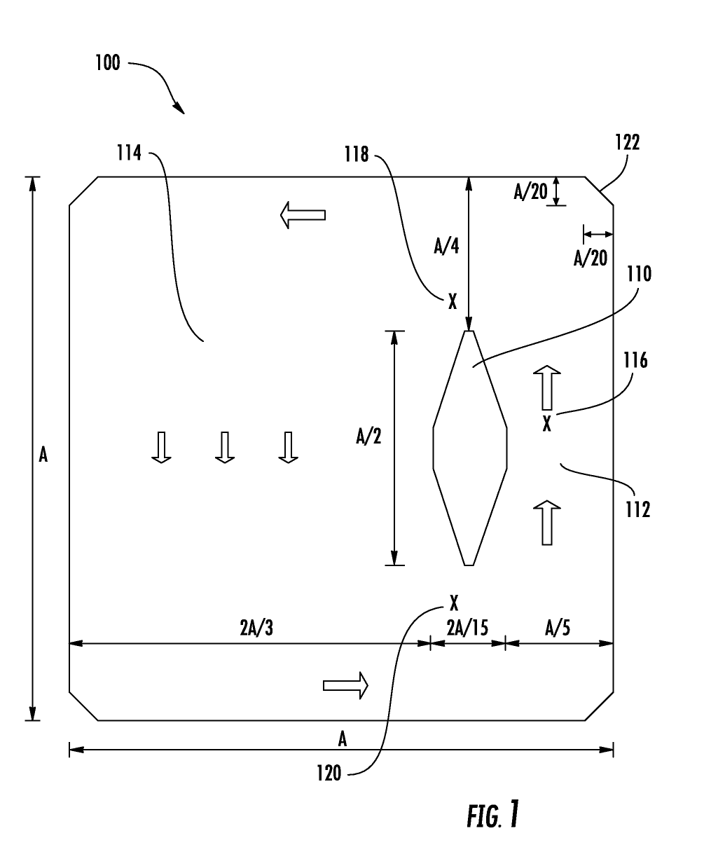

FIG. 1 depicts an example embodiment for an LF pond. The LF pond 100 is for cultivating algae and is generally square-shaped with an aspect ratio of length to width of between 0.7:1 to 2:1. In another embodiment the aspect ratio of length to width may be between 0.8:1 to 1.2:1. In a further embodiment, the pond shape may be rectangular with an aspect ratio of less than 3. Referring to FIG. 1, sample dimensions are labeled on LF pond 100 assuming, in this embodiment, the LF pond 100 is squared shaped and the length of one side of the LF pond 100 is A. Other sample dimensions on the figure are based on A.

The overall size of the pond is at least 5 acres and in further embodiments, the overall size of the pond is at least 20 acres, 40 acres, 80 acres, 100 acres or 160 acres. In other embodiments is the pond is between 5 acres and 50 acres, between 50 acres and 160 acres, between 160 acres and 320 acres, between 320 acres and 500 acres, or between 500 acres and 640 acres. The average pond depth is at least 15 inches. In another embodiment, the average pond depth is 8 inches. In additional embodiments, the average pond depth is between 6 inches and 10 inches, between 8 inches and twelve inches, or between twelve inches and 16 inches. The LF pond 100 may be lined or unlined. The LF pond 100 includes an island 110.

The island 110 has a first side and a second side and is positioned in the LF pond 100 to create a first channel or throat 112 adjacent the first side of the island 110 and a second channel or open field 114 adjacent the second side of the island 110. The width ratio of the first channel or throat 112 to the second channel or open field 114, is a design parameter that can be utilized to decrease energy consumption and minimize no-to-low flow areas or deadzones. By optimizing the configuration and placement of the island 110, the size of the deadzones is reduced. The second channel 114 is at least three times wider than the first channel 112. In other embodiments, the second channel 114 may be at least five times wider than the first channel 112, or at least seven times wider than the first channel 112.

The island 110 is substantially elongated diamond shaped or oval shaped and includes substantially a pointed tip at both the first end and the second end. The length of the island is at least six times larger than the width of the island. Moreover, the ratio of the length of the island to the width of the island is approximately 3.75. In another embodiment, the ratio of the length of the island to the width of the island is between 2 and 5. In further embodiments, the shape of the island may be rectangular, circular, square or any arbitrary shape. The island 110 may be constructed from dirt or sand or other suitable materials. Other suitable materials include, but are not limited to, modular barriers, for example, concrete blocks, water filled plastic blocks, expanded foam core blocks, metal barriers and the like. Such modular barriers proved for easy island remove and reinstallation during pond maintenance. Berms may be used to construct the island 110 and may have straight or curved lines.

An energy input means or flow motivation system 116 is located in the first channel or throat 112 for moving fluid around the island 110 through the first channel or throat 112 and through the second channel, which is also referred to as an open field, 114. The size and cost of the flow motivation system is minimized by decreasing the width of the throat. The energy input means or flow motivation system 116 may be in a single area in the throat 112, which may be a point or along a single axis. Said single axis may extend the width of said first channel or any portion thereof. The energy input means or flow motivation system 116 may be a pump such as submersible or centrifugal, paddlewheels, fan, nozzle, jets or the like, or a combination of any of these. For example, the flow motivation system 116 may be one submersible pump. In another embodiment, the flow motivation system 116 may be several paddlewheels along a single axis, across the throat 112 of the LF pond 100. In a further embodiment, the flow motivation system 116 may be multiple nozzles along the axis across throat 112 or multiple fan style pumps in lieu of nozzles.

The flow motivation system 116 generates a hydraulic head at the throat 112. The difference in hydraulic head between the flow induced in the throat 112 and the open field 114 causes the direction of induced flow to move towards the open field 114. Since the flow velocity is higher in the throat 112, the induced flow fills the open field 114 quickly and efficiently. Referring to FIG. 1, the arrows indicate the flow pattern.

The flow of fluid in the LF pond 100 varies from point to point and does not have a constant velocity throughout the pond. A consistent flow is generated throughout the second channel or the open field 114. The average fluid velocity is at least 3 centimeters/second. In another embodiment, the average fluid velocity is 5-10 centimeters/second. In still other embodiments the average fluid velocity is between 2 centimeters/second and 5 centimeters/second, between 3 centimeters/second and 5 centimeters/second, between 5 centimeters/second and 8 centimeter/second, or between 8 centimeters/second and 10 centimeters/second. The LF pond 100 is designed for slower flow velocities but there is no fundamental restriction for increased velocities beyond the needed energy demand to support higher velocities. The flow circulates from the flow motivation system 116 in the throat 112 to the open field inlet 118, to the open field 114, to the open field outlet 120 and back to the throat 112 where the flow is increased by subsequent passes through the flow motivation system 116.

The flow in the LF pond corners can be considered deadzones. There may be significant algae settling in these deadzone regions, as well as an accumulation of pests and/or bacteria. In order to have the LF pond flow more uniformly distributed throughout the pond volume, the corners of the LF pond 100 may be cut forming clipped corners 122. The cut may be a cut forming an equilateral triangle which is 1/20 of the LF pond side. Therefore, the LF pond 100 may be configured with clipped corners. Furthermore, by hydraulically optimized the size and position of the island 110, the extent of deadzones are reduced with limited improvement of flow in the deadzone area.

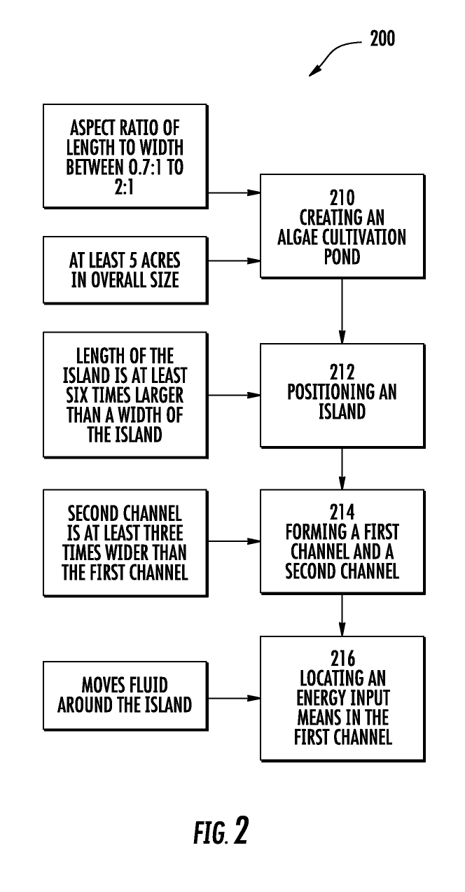

FIG. 2 is an example flowchart for a method for circulating fluid in an algae cultivation pond. The method 200 begins at step 210, by creating an algae cultivation pond. The pond has an aspect ratio of length to width between 0.7:1 to 2:1, and is at least 5 acres in overall size. At step 212, an island with a first side and a second side is positioned in the pond. The length of the island is at least six times larger than a width of the island. At step 214, a first channel adjacent the first side of the island and a second channel adjacent the second side of the island is formed. The second channel is at least three times wider than the first channel. At step 216, an energy input means is located in the first channel for moving fluid around the island through the first channel and through the second channel.

The advantages of LF ponds for algae cultivation compared to traditional algae cultivation ponds such as center pivot ponds, serpentine ponds, no-flow (or unmixed) rice field ponds or raceway ponds are numerous. The design of the LF pond has fewer pressure-bearing walls as compared to other ponds, such as serpentine ponds. This reduces the cost of construction as well having less drag from walls. The LF pond is designed for a minimum size of flow motivation equipment per acre. There is a minimum amount of hydraulic head required for moving water in the pond per acre. Additionally, there is a minimum amount of head required for moving water in the pond per acre.

The LF pond has very low head losses at a constant velocity and therefore lower energy requirements and costs for flow motivation per acre. For example, in this implementation, the head loss may vary as little as 1-3 inches. In contrast, in other algae cultivation ponds of the same acreage, such as a serpentine type pond, the head loss may vary 1-3 feet. The reduction in the amount of head loss for the LF pond is achieved by sizing and configuring the first channel, or throat, and the second channel, or open field, as described herein, while the induced flow by the energy input means in the throat section is injected into an open field. The low amount of head loss for the LF ponds provides an opportunity for the cost requirements for capital as well as the cost requirements for operation to be significantly lower than traditional algae cultivation ponds such as raceway ponds. In addition, ponds with large head losses may require multiple or more motivation systems (i) to be provide at multiple locations, (ii) or to be operated at variable depths, ranging from as low as 10 inches up to 1-2 feet.

The LF pond may be built with minimum amount of berms per acre and these berms are designed without using a curved berm which is less expensive to construct. The LF pond unique configuration provides consistent flow in the throat and open field with minimum deadzone area per acre. The design of the LF pond allows for fast flow at the throat leading to high energy efficiency. Overall, the LF pond has low operating costs per acre.

Through simulation, different sized LF ponds as well as virtual velocities and pond geometries were designed. Ponds were simulated with aspect ratios of 1.0 to 1.2. The smallest LF pond dimensions simulated were 27 feet by 27 feet. The largest LF pond dimensions simulated was 160 acres, measuring 0.5 mile×0.5 mile square. FIG. 3 is a table for example simulated cases of various sized LF ponds.

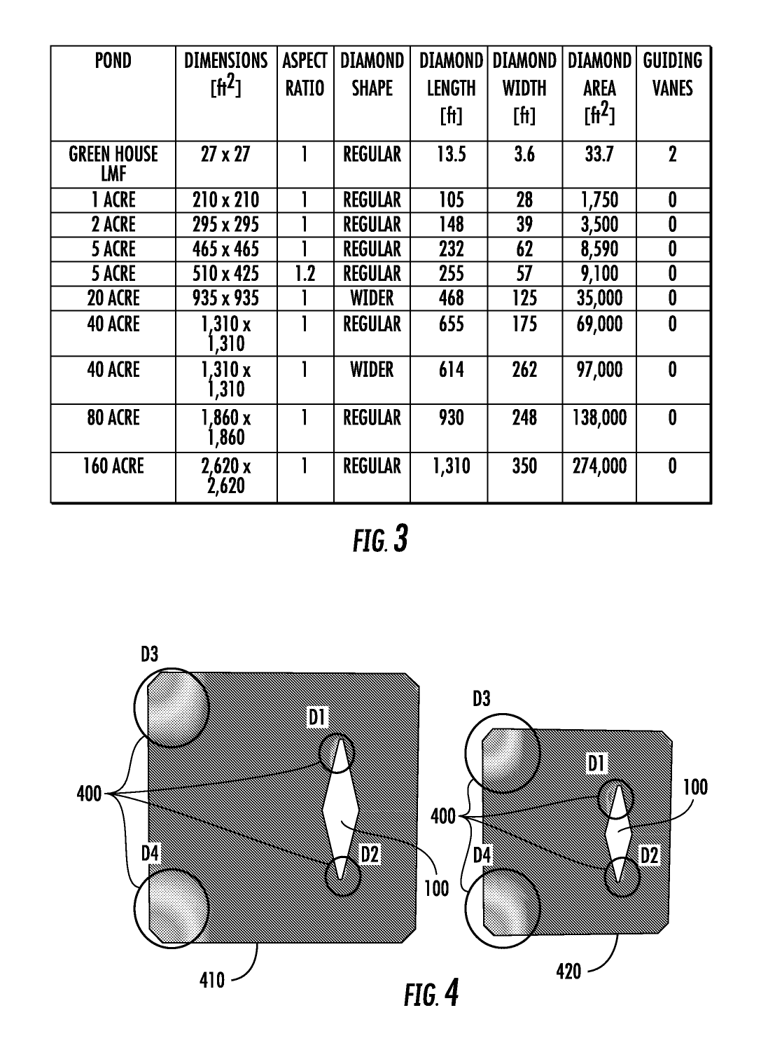

FIG. 4 illustrates an example simulation for potential deadzones in LF ponds. A 160 acre LF pond 410 with an island 110 is compared to an 80 acre LF pond 420 with an island 110. The average velocity in both of the LF ponds 410 and 420 was less than 5 centimeters/second. The total percentage of deadzones 400 does not change for a 160 acre LF pond to an 80 acre LF pond. This proved counterintuitive to the idea that a larger pond would have larger areas of deadzones than a smaller pond. It is also counterintuitive to the idea that a larger pond has more stagnation areas than a smaller pond.

Harvesting capital and operating costs of algae cultivation for harvesting techniques may be related to the harvesting flow density. Therefore, by increasing the cell density of harvesting flow, the harvesting volume is decreased and thus, smaller harvesting units may be used as well as reducing harvesting energy.

The LF pond velocity variations as well as various fast-flow and low to-no-flow regions, or deadzones, creates opportunities to combine harvesting and cultivation of algae strategies. The combination of harvest and cultivation activities may significantly reduce the cost requirements for capital as well as the cost requirements for operation of the LF pond. The deadzones areas along corners of the LF pond allow the accumulation of algae. By designing a harvest sub-pond in these areas, harvesting and cultivation activities of algae may be combined.

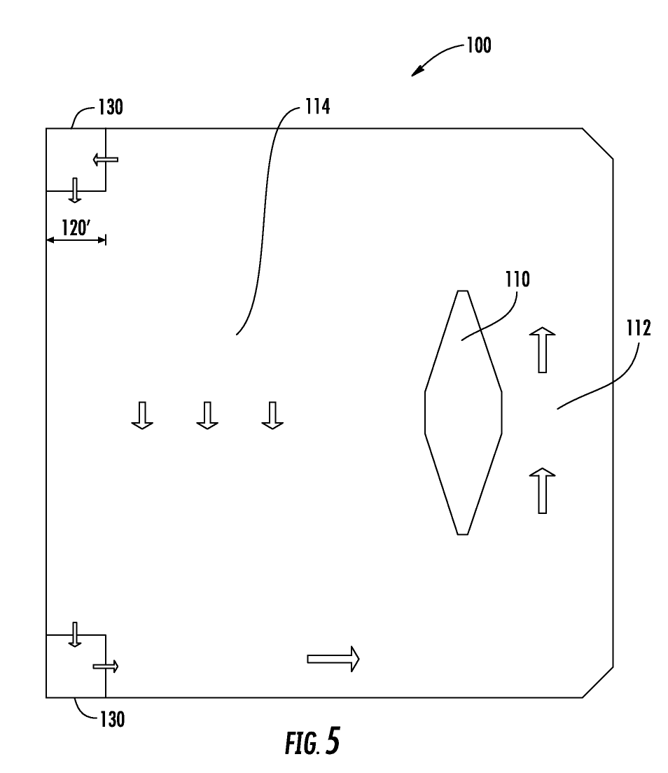

A sub-pond located at the corner(s) of the LF pond may be implemented to accumulate biomass. FIG. 5 shows an example embodiment of a harvest sub-pond located in the LF pond. The LF pond has the same configuration as described herein with the island 110, the throat 112 and the open field 114. The LF pond 100 may be configured to include harvesting corners for algae. The harvesting corners are a semi-isolated region promoting advanced settling and concentration of algae. For example, harvesting corners or the sub-pond 130 is located in two of the corners of the LF pond 100 but note that in this embodiment, the corners are not clipped in order to promote algae settling in these deadzone regions. In another embodiment, the corners may be clipped as described herein.

FIG. 6 is an example embodiment of a harvest sub-pond. A weir is a barrier across a body of water designed to alter its flow characteristics. These are smaller than a conventional dam, and causes water to pool behind them while also allowing the water to flow steadily over their tops. The harvesting corners, or sub-pond 130, is comprised of weirs. The weirs create semi-isolated regions from a main body of the pond, and have a height of approximately 2 inches less than the average depth of the main body of the pond. Fluid with an algae culture is exchanged between the main body of the pond and the harvesting corners over a top of the weirs.

Two weirs 132 are located in the corner of the LF pond 100 which are the no-to-low flow corners. By locating the weirs 132 here, a semi-isolated region, or sub-pond 130 is formed. The density of an algae culture entering the harvesting corners, or sub-pond 130, is greater than the density of the algae culture exiting the harvesting corners, or sub-pond 130. For example, algae culture having nominal LF pond density enters the sub-pond 130 and less dense algae culture exits from the sub-pond 130. The weirs 132 separate the no-to-low flow corners or sub-pond from the body of the LF pond 100 and promote advanced settling and concentration of algae in these areas.

Referring to FIG. 6, sample dimensions are detailed on the figure. For example, the LF pond 100 is 160 acres. The size of the sub-pond 130 is 30 feet by 30 feet. The height of the weirs 132 may be 1-2 inches shorter than the average depth of the main body of LF pond 100. In one embodiment, the average pond depth is approximately 9 inches and the height of the weirs 132 is approximately 7 inches. The exchange of algae culture between the LF pond 100 and the sub-pond 130 is through the top 1-2 inches of the available water column at the sub-pond inlet 134 over the weir. The exchange of the top 1-2 inches of water contains considerably less sand and heavy debris when compared to the water in the body of the LF pond 100. The sub-pond outlet 136 return of water to the LF pond 100 is also over the top 1-2 inches of water column over the weir 132. The sub-pond 130 may be lined or unlined.

The algae culture exiting the sub-pond 130 by the sub-pond outlet 136 is more dilute than the water in the LF pond 100 due to the increased residence time of the algae culture in the sub-pond 130 and the stagnant nature of flow in this area. The algae culture in the sub-pond 130 may be stagnant and after some time, settles in these areas. The 1-2 inch gap between the top of the weir 132 and the top of the average depth of the LF pond 100 allows diluted algae culture to exit the sub-pond 130 through the sub-pond outlet 136 and over the weir 132. The density of the algae culture in the sub-pond 130 slowly increases over time to a point where it may be harvested via a pump. The transfer of the algae culture from the sub-pond 130 to a main harvesting unit may be performed as fast as possible to prevent dilution of this concentrated algae culture, due to incoming flow over the sub-pond inlet 134. In one embodiment, the LF pond is unlined as is the sub-pond. In another embodiment the pond is unlined and the sub-pond is lined. When the algae culture circulates in an unlined LF pond and enters the sub-pond, large sediment and debris accumulates just below the sub-pond inlet. The settled algae culture in the sub-pond may be cleaner than the algae culture in the LF pond.

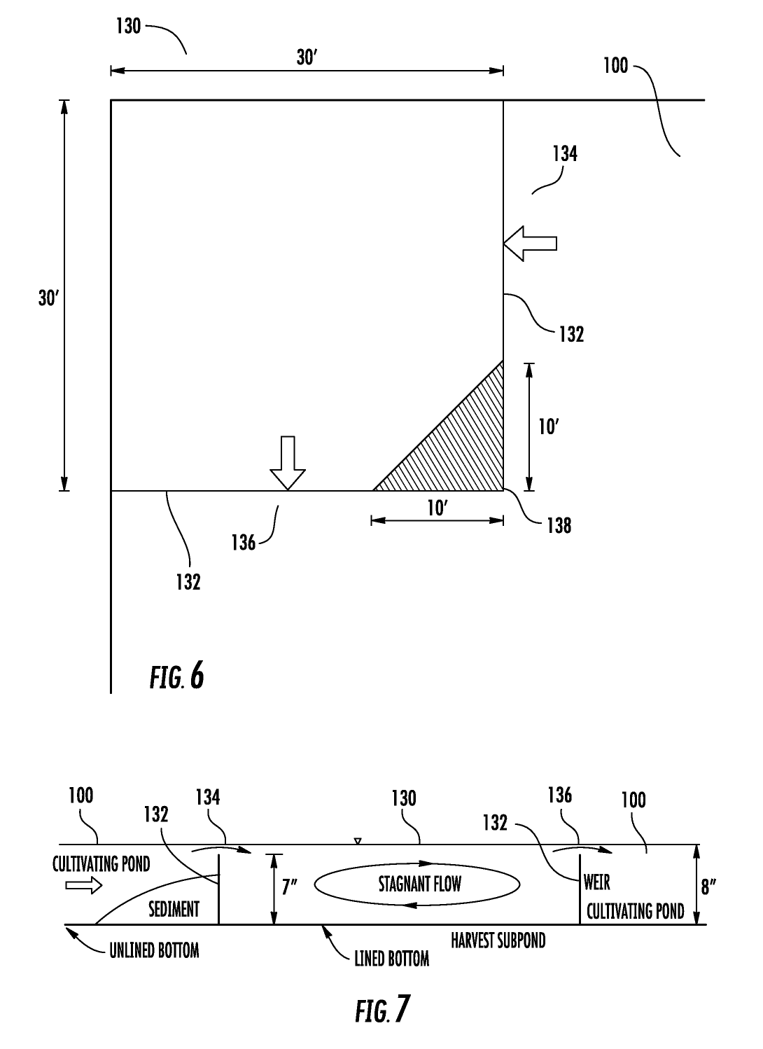

FIG. 7 illustrates an example embodiment of the flow in a harvest sub-pond. In this example embodiment, the water with algae culture flows according to the arrows detailed in the figure. For example, the water with algae culture from an unlined LF pond 100 contacts the weir 132 and sediment collects against the weir 132. The weir 132 has a height of approximately 7 inches and the average pond depth is approximately 8 inches. The water with algae culture then enters the sub-pond 130 by passing over the weir 132 through the sub-pond inlet 134. The sub-pond 130 has a lined bottom and stagnant flow. The algae culture eventually settles to the bottom of the sub-pond 130. The water with any remaining algae culture, then contacts a second weir 132 and passes over the top of the second weir 132 through the sub-pond outlet 136 and back to the LF pond 100.

In another embodiment, the sub-pond 130 includes a sub-pond flow guiding island 138. Referring to FIG. 6, the sub-pond flow guiding island 138 may be located in the corner of the sub-pond 130 where the two weirs 132 join together. In this example, the sub-pond flow guiding island 138 is triangular shaped with a base and height of 10 feet. The sub-pond flow guiding island 138 aids in directing flow to the sub-pond 130 area.

In another embodiment, an algae froth floatation harvest method that utilizes the observed foam floating in the LF pond during cultivation may be used. A low pressure zone created by the energy input means or flow motivation system, such as a jet nozzle, coupled with the over-saturated dissolved gas, for example, carbon dioxide (CO2), air, nitrogen or the like, within the algae culture creates foam on the top of the LF pond. A top-floating barrier or boom may be used to gather the algae foam on top of the pond. The density of the algae foam slowly increases over time to a point where it may be harvested via a pump. The boom allows full flow beneath the top layer of foam and may have little impact on the flow throughout the LF pond.

FIG. 8 is an example embodiment of an algae froth floatation harvest method in an LF pond. The LF pond 100 is configured as discussed herein with an island 110, throat 112, open field 114 and harvest sub-pond 130. Sample dimensions are detailed. A boom 148, a floating weir box 150 and a pump system 152 may be used to remove the algae foam 154 (also referred to as harvestable foam) at harvest. In one embodiment, the harvesting area with the boom 148 and floating weir box 150 may be located in the throat 112. By generating foam as part of a harvest method, the amount of harvested volume may be reduced. The size of the harvest unit as well as the piping operating expenses may also be reduced due to the increased density of algae and subsequent reduction of required harvest water. The algae foam 154 may be dewatered using dewatering tanks 156 at the time of harvest. The dewatering tanks 156 may be a series of flow-through baffled tanks.

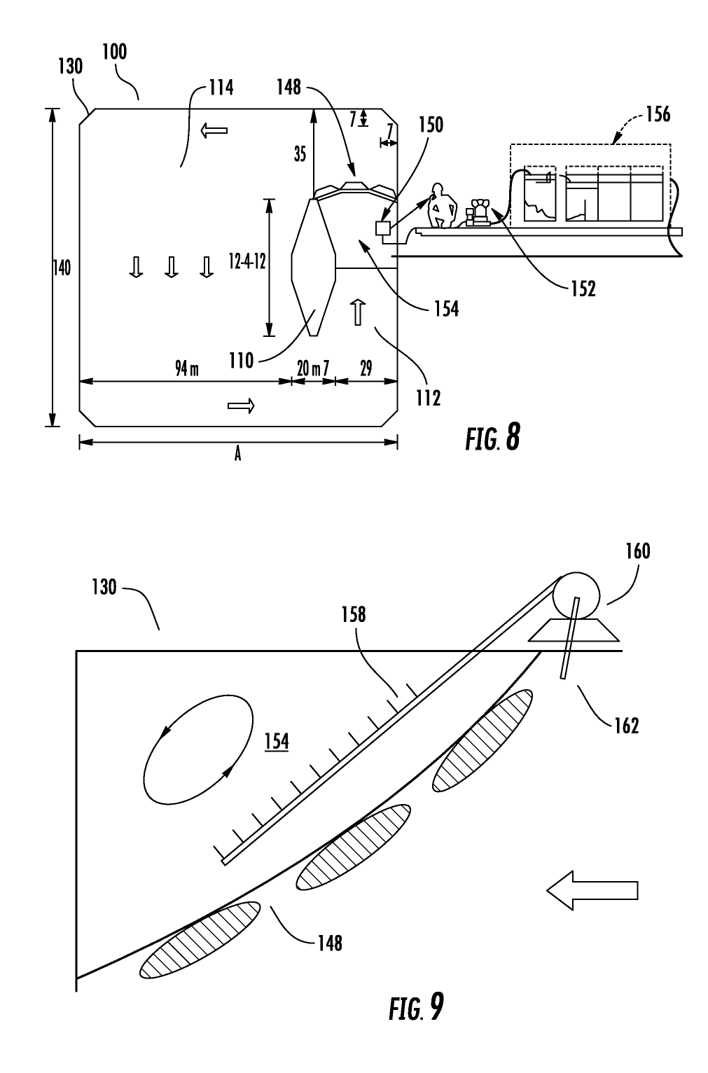

In another embodiment, the location of the harvested area may be located in the no-to-low flow corners of the LF pond, or the harvest sub-pond 130 area. FIG. 9 is an example embodiment of an algae froth floatation harvest method in a harvest sub-pond. In this implementation, the boom 148, a mobile jet nozzle system 158, a pump 160 and suction 162 are located in one of the no-to-low flow corners (sub-pond 130). This is an area where algae settling occurs. When utilizing the boom 148 with a mobile jet nozzle system, the settled algae may be re-suspended while inducing foam in these areas. The algae foam 154 may then be removed. The algae froth floatation harvest method may harvest a tenth less volume but is ten times more concentrated. The floating weir box 150 used for transferring the algae foam to the main harvesting unit is a simple, effective surface skimmer that may be adjusted for varying depths with a flow rate of suction between 5 to 95 gpm.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the invention. Thus, it is intended that the present subject matter covers such modifications and variations.

1. A system for circulating fluid in an algae cultivation pond, the system comprising:
an algae cultivation pond having an aspect ratio of length to width between 0.7:1 to 2:1, the pond being at least 5 acres in overall size;
an island with a first side and a second side, the island being positioned in the pond to create (i) a first channel adjacent the first side of the island and (ii) a second channel adjacent the second side of the island; and
an energy input means located in the first channel for moving fluid around the island through the first channel and through the second channel;
wherein the second channel is at least three times wider than the first channel; and
wherein a length of the island is at least six times larger than a width of the island.

2. The system for circulating fluid in the algae cultivation pond of claim 1, wherein the energy input means is in a single area in the first channel, the single area being a point or along a single axis.

3. The system for circulating fluid in the algae cultivation pond of claim 1, wherein the energy input means is a pump, paddlewheel, fan, nozzle or jets.

4. The system for circulating fluid in the algae cultivation pond of claim 1, wherein an average fluid velocity is at least 3 centimeters/second.

5. (canceled)

6. (canceled)

7. (canceled)

8. The system for circulating fluid in the algae cultivation pond of claim 1, wherein the pond has an aspect ratio of length to width between 0.8:1 to 1.2:1.

9. (canceled)

10. The system for circulating fluid in the algae cultivation pond of claim 1, wherein the island is substantially elongated diamond shaped or oval shaped.

11. The system for circulating fluid in the algae cultivation pond of claim 1, wherein the island includes substantially a pointed tip at both a first end and a second end.

12. The system for circulating fluid in the algae cultivation pond of claim 1, wherein a ratio of the length of the island to the width of the island is approximately 3.75.

13. The system for circulating fluid in the algae cultivation pond of claim 1, wherein a ratio of the length of the island to the width of the island is between 2 and 5.

14. The system for circulating fluid in the algae cultivation pond of claim 1, wherein the pond is configured to include harvesting corners for algae;
wherein the harvesting corners are a semi-isolated region promoting advanced settling and concentration of algae; and
wherein a density of an algae culture entering the harvesting corners is greater than the density of the algae culture exiting the harvesting corners

15. (canceled)

16. (canceled)

17. The system for circulating fluid in the algae cultivation pond of claim 14, wherein the harvesting corners are comprised of weirs, the weirs (i) creating semi-isolated regions from a main body of the pond, and (ii) having a height of approximately 2 inches less than an average depth of the main body of the pond;
wherein fluid with an algae culture is exchanged between the main body of the pond and the harvesting corners over a top of the weirs.

18. A method for circulating fluid in an algae cultivation pond, the method comprising:
creating an algae cultivation pond having an aspect ratio of length to width between 0.7:1 to 2:1, the pond being at least 5 acres in overall size;
positioning an island with a first side and a second side in the pond;
forming (i) a first channel adjacent the first side of the island and (ii) a second channel adjacent the second side of the island; and
locating an energy input means in the first channel for moving fluid around the island through the first channel and through the second channel;
wherein the second channel is at least three times wider than the first channel; and
wherein a length of the island is at least six times larger than a width of the island.

19. The method for circulating fluid in the algae cultivation pond of claim 18, wherein the energy input means is in a single area in the first channel, the single area being a point or along a single axis.

20. The method for circulating fluid in the algae cultivation pond of claim 18, wherein the energy input means is a pump, paddlewheel, fan, nozzle or jets.

21. (canceled)

22. (canceled)

23. (canceled)

24. The method for circulating fluid in the algae cultivation pond of claim 18, wherein the pond has an aspect ratio of length to width between 0.8:1 to 1.2:1.

25. (canceled)

26. The method for circulating fluid in the algae cultivation pond of claim 18, wherein the island is substantially elongated diamond shaped or oval shaped.

27. The method for circulating fluid in the algae cultivation pond of claim 18, wherein the island includes substantially a pointed tip at both a first end and a second end.

28. The method for circulating fluid in the algae cultivation pond of claim 18, wherein a ratio of the length of the island to the width of the island is between 2 and 5.

29. The method for circulating fluid in the algae cultivation pond of claim 18, wherein the pond is configured to include harvesting corners for algae;
wherein the harvesting corners are a semi-isolated region promoting advanced settling and concentration of algae; and
wherein a density of an algae culture entering the harvesting corners is greater than the density of the algae culture exiting the harvesting corners.

30. (canceled)

31. (canceled)

32. The method for circulating fluid in the algae cultivation pond of claim 29, wherein the harvesting corners are comprised of weirs, the weirs (i) creating semi-isolated regions from a main body of the pond, and (ii) having a height of approximately 2 inches less than an average depth of the main body of the pond;
wherein fluid with an algae culture is exchanged between the main body of the pond and the harvesting corners over a top of the weirs.
